(19) United States
(12) Patent Application Publication
(10) Pub. No.: US 2010/0049310 A1
(43) Pub. Date: Feb. 25, 2010

(54) METHOD FOR COATING A STENT

(75) Inventors: Eckhard Quandt, Heikendorf (DE); Holger Rumpf, Reutlingen (DE); Christiane Zamponi, Kiel (DE); Michael Koch, Bonn (DE)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: ACANDIS GmbH & Co. KG, Pfinztal (DE)

(21) Appl. No.: 12/279,647

(22) PCT Filed: Feb. 15, 2007
(86) PCT No.: PCT/EP2007/001329

§ 371 Date: Jul. 31, 2009

(30) Foreign Application Priority Data
Feb. 15, 2006 (DE) 10 2006 007 231.6

(51) Int. Cl. A61F 2/06; B05D 7/00

(52) U.S. Cl. 623/1.46; 427/2.25

(57) ABSTRACT

Method for applying a coating layer to a tubular intraluminal implant, in particular to a vascular support (stent), where the surface of the implant is perforated by a plurality of apertures, and where the coating layer is produced by deposition of material onto the surface of the implant. The implant is first pushed onto a cylindrical holder 4, a sacrificial material, in particular copper, is then deposited onto the surface of the implant until the deposited sacrificial material almost entirely fills the apertures, the coating layer is then deposited onto the surface of the implant provided with sacrificial material, and then the cylindrical holder 4 and the sacrificial material situated in the apertures 3 are removed.

### CROSS-REFERENCE TO RELATED APPLICATION

The present application claims the benefit of priority of International Patent Application No. PCT/EP2007/001329, filed Feb. 15, 2007 which application claims priority of German Patent Application No. 10 2006 007 321.6, filed Feb. 15, 2006. The entire text of the priority application is incorporated herein by reference in its entirety.

### FIELD OF THE DISCLOSURE

The present disclosure relates to a method for applying a preferably metallic coating layer to a tubular intraluminal implant, in particular to a vascular support (stent), wherein the surface of the implant is perforated by a plurality of apertures, wherein the coating layer is produced by deposition of material onto the surface of the implant. The disclosure also relates to the implant coated with this method.

### BACKGROUND

Stents, which resemble small tubular mesh frameworks are implanted into vessels for the therapeutic expansion of said vessels, for example the cardiac coronary vessels or for the prevention of recurrent narrowing, and when in place, stabilise the vessel walls. The mesh-like structure of stents is necessary so that they can be introduced in a folded condition into the vessel before being unfolded and thereby placed against the wall of a vessel from inside. A distinction needs to be made between self-unfolding stents, which relax in spring-like manner and thereby unfold once a surrounding plastics sleeve has been withdrawn in situ. In contrast thereto, other stents are placed and expanded using a balloon catheter.

A problem with previously known stents is that their mesh-like structure can lead to irritation of the tissue and thereby to a certain degree of injury to the vessel inner wall. When this endogenous inflammation heals, tissue is formed which penetrates the apertures in the surface and grows into the lumen. In approximately 20% of cases, the vessel is again narrowed by this process of restenosis such that a repeated intervention becomes necessary.

“Covered” stents are known, which are covered with a net made from PTFE, wherein the covering is intended to contribute to reducing restenosis. A further advantageous effect of the covering is that the thrombotic material situated in the vessels is pressed by the more closed surface of the stent against the inner wall of the vessel and is firmly held there. The introduction of thrombotic material into the bloodstream by implantation of the stent can therefore be largely avoided, so that the danger of spreading this material in the circulatory system and the risks associated therewith are reduced.

### SUMMARY OF THE DISCLOSURE

It is an aspect of the disclosure to provide a method for coating an implant of this type which can be easily carried out and is suitable for the production of such implants on a large scale. It is a further aspect of the disclosure to improve a covered stent of this type such that, given economical production and easy deployment, it contributes to the avoidance of restenosis and fixes thrombotic material with a high level of efficiency.

The essential concept of the disclosure lies therein that the apertures in the surface of the implant are filled with a sacrificial material and that thereby a temporary smooth surface is created. A covering material which forms a film-like coating can subsequently be deposited thereon. The sacrificial material is then removed again, at least from the apertures, most favourably from inside, so that the functionality of the implant is restored again. The covering film which has an adequate hold on the surface formed by the mesh remains behind. It must be ensured that the film has adequate flexibility, since it has to be able to conform suitably to the expansion of the implant in the vessel. Flexibility of this type is achieved, in particular, with a net-like structure, wherein the dimension of the mesh should be an order of magnitude smaller than that of the apertures. In the description which follows, the term ‘stent’ is taken to be synonymous with tubular implants of this type. The disclosure is also applicable to vascular implants such as filters, stent grafts or to occlusion instruments or occluders.

It is essential, according to the inventive solution, that the film-like covering is bonded to the individual webs of the mesh structure of the stent or implant not merely at points but at least in sections with uninterrupted material contact. In the case of a tubular stent or implant, this material contact is formed individually between the surface of the mesh structure comprising the outside of the stent or implant and the preferably metallic, film-like covering.

The essential steps of the method according to the disclosure involve firstly pushing an existing stent onto a cylindrical holder. Once it has been fixed in this manner, in a further step, sacrificial material that is later to be removed, in particular copper, is deposited over the mesh of the stent. This deposition is continued until the sacrificial material almost fills the apertures and the mesh structure is closed. As will be described, it may be advantageous to treat the sacrificial material such that it is at least partially removed from the outside before, in another step, the coating layer is deposited on the surface of the stent that is thereby provided.

Depending on how strong the bond between the mesh of the stent and the coating layer is to be, during the treatment, more or less sacrificial material is removed from outside. It may be advantageous if, by means of the treatment, the mesh structure of the stent is laid bare to such an extent that the coating layer is able to bond thereto. If, on the other hand, a layer of sacrificial material remains between the mesh and the coating layer, the covering formed on the stent by the coating layer will be displaceable, due to the lack of any bonding.

Finally, the sacrificial material which is found between the coating layer and the stent and, above all in the apertures, is removed.

A substantial advantage of the method according to the disclosure and the stent resulting therefrom is that the ingrowth of tissue into the vessel, that is, the formation of a restenosis can be at least drastically reduced if not entirely avoided. The method can be applied to all known types of stent (or vascular implants). To that extent, for example, all the commercially available stents can be improved with this method. Naturally, the method according to the disclosure is also usable for vascular implants, for example, filters, stent grafts and occlusion instruments in order to improve these medical devices, particularly with regard to their tolerability in the body.

It should be noted at this point that the embodiments, advantages and effects of the method according to the disclosure described herein in relation to improvement of a stent can be applied or achieved in similar manner with vascular implants, for example, filters, stent grafts or occlusion instruments.

A further advantage that can be gained with the method according to the disclosure is to be found therein that—due to the complete covering of the stent (or the vascular implant) with the coating layer, the possibility also exists that, by means of suitable treatment of the covering and, in particular, subsequent structuring of the covering, a ‘covered’ stent or stent graft with a very fine mesh size, which is ideal for covering aneurysms can be produced. The possibility therefore exists, in particular, of adapting the stent or the vascular implant specifically to the patient in that the scope and/or the type of treatment of the completely covered stent (or implant) are adapted to the individual case. It should be noted in this context that the ‘covering’, that is, the coating comprising the covered stent, is not usually subjected to excessive forces. However, it is advantageous if the covering is as thin as possible in order to enable implantation and possibly also removal of the medical device with, for example, one installing and removing tool having the smallest possible diameter. After positioning of the covered stent or stent graft into the body of the patient, the covering can be expanded with a conventionally produced stent which has the relevant macroscopic wall thickness and web widths in order to ensure the mechanical stability of the implant.

With regard to a medical device (stent, vascular implant) produced with the method according to the disclosure, a further advantage is that with this medical device—in particular due to the covering film, which has an adequate hold on the surface formed by the mesh—a continuous bond with material contact between the mesh structure of the conventionally produced implant (stent) and the covering is produced. By this means, it is achieved that the medical device according to the present disclosure is significantly less thrombogenic than conventional medical devices, such as stents or occlusion instruments that are produced by a conventional method.

It is particularly advantageous if the surrounding coating layer is produced as a film made from metal, in particular a nickel titanium alloy (NiTi), for example Nitinol. Metal has the advantage in this regard that it can be applied by vapor deposition or sputter deposition at an adjustable thickness and a high surface quality. In addition, a chemically and biologically inert alloy, such as the aforementioned Nitinol, which does not impair the functioning of the organism, can be selected. A further significant advantage of the covering metal film is its stability, as a result of which a particularly planar surface over the mesh-like structure is achieved. With the stable and planar surface of the stent, good fixation of any thrombotic material present on the inner wall of the vessel can be assured.

Alternatively or additionally to the above advantageous embodiment of the method according to the disclosure wherein the covering coating layer is produced as a film made from metal, in particular a nickel-titanium alloy (NiTi), for example Nitinol, it is also conceivable for a covering film to be formed by vapor deposition of biodegradable metal films, particularly made from iron, iron alloys, magnesium or magnesium alloys, on the mesh of the medical device, said film at least partially or fully decomposes and therefore disappears after a previously determined time. This allows the medical device to be used in highly application-specific manner.

In a preferred embodiment of the solution according to the disclosure, it is provided that the (conventionally produced) implant to be processed using the method according to the disclosure or the (conventionally produced) stent to be processed using the method according to the disclosure and/or the metal film applied or to be applied according to the disclosure onto the mesh of the medical device (implant, stent) is made from a preferably biodegradable metal, in particular iron, iron alloys, magnesium or magnesium alloys.

Naturally, it is also conceivable that the metal film is formed at least partially from an X-ray opaque material, for example gold, platinum, niobium or tantalum. These metals can also be introduced into the metal film by sputter deposition, so that the metal film is made, for example, from the ternary alloys NiTiTa or NiTiNb.

In an advantageous embodiment, the mesh-like structure of the stent is filled with a metal as a sacrificial material, in particular with copper, by means of a galvanic method. Herein, the stent made, in particular, from Nitinol is mounted on a tube of suitable material, in particular therefore copper, and galvanically coated with the metal in a bath. The process is stopped when the electrochemically deposited layer largely, in particular completely, covers the mesh structure. The surface thereby produced is then advantageously treated with a soft abrasive paper on a lathe to remove the excess metal. Thereafter, the ‘blocked’ mesh of the stent in whose apertures the copper has been deposited, is subjected to sputter deposition like an ordinary tube with metal, in particular with superelastic NiTi before the copper core is removed with a selective acid treatment.

For the sacrificial material, Au, Cr, FeCo or the like can be selected and this material is applied to a substrate tube made from sacrificial material, preferably onto Cu, but optionally also onto Au, Cr or FeCo. It should be noted that the substrate and the galvanically deposited layer can be selectively etched against the stent made from NiTi, wherein the sacrificial substrate should be softer than the material of the stent, in order that no stent material and only small amounts of the NiTi are removed by the abrasion of the sacrificial substrate. NiTi is harder than copper.

The use of NiTi, in particular Nitinol, for the production of stents is known. As described, NiTi is also particularly preferable for the covering of the stent. Aside from the biocompatibility, the essential feature of these alloys is, above all, their shape memory together with their superelasticity. NiTi is a preferred representative of the alloys with shape memory. It is at least 8% pseudoelastically deformable, corrosion-resistant and has high strength. Within a temperature interval and above a characteristic pre-tension of a few hundred MPa, the stress-strain curve has a plateau. In this strain region, a phase change takes place within the material wherein, under loading, the cubic face-centered austenite is converted to monoclinic martensite. Depending on the stress applied, the stress-induced martensite can detwin and this enables deformation of the material within the plateau at a constant counter-force. Herein, by means of the phase transformation, strains of up to 8% in the strain-induced martensite can be absorbed without plastic deformation taking place. On removal of the load on the martensite, it reverts again into the starting condition of the austenite.

As a result of its biocompatibility, NiTi is frequently used in medical technology. The superelastic properties of NiTi are advantageous in medical instruments such as catheters and particularly for the positioning of stents which are subject to severe deformation during their deployment in the body. Tissue spreaders with superelastic properties, in particular, have the advantage that they cause less damage to the tissue than spreaders made from other materials. The shape memory effect of NiTi can be utilised in stents in particular. They are deformed in a martensitic condition at room temperature. If the austenitic high temperature phase is stable at body temperature, the implant assumes its original form. Thus, folded stents can unfold independently in the body. Naturally, this also applies for a covering made from NiTi, which shows the same elastic properties. To this extent, NiTi stents of this type coated with NiTi make ideal implants for vessel expansion and vessel support.

Production of the thin coating layer with superelastic properties is advantageously carried out by a physical deposition process, preferably by cathode evaporation or sputter deposition. Sputter deposited films of this type can show superelastic behavior at body temperature, given a strain of more than 6.5% at, for example, a plateau stress of 400 MPa or less.

If the flexibility is not permitted by the material itself or if the intrinsic flexibility of the coating layer is insufficient, it is also advantageous to provide apertures therein, although these should be smaller than those of the stent. The structuring of the coating layer is advantageously carried out by means of photolithographic, laser-supported or other processes. It is thereby possible to create a coating layer with a fine net-like structure and high elasticity. A particularly preferred application of the method is thus the optimising of NiTi cylinder structures for endovascular or neurovascular NiTi stents, wherein by means of the coating layer, a reduction in the pore size is possible, while maintaining the superelastic properties of the supporting structure made, in particular, from laser-cut NiTi.

### BRIEF DESCRIPTION OF THE DRAWINGS

The disclosure will now be described in greater detail by reference to FIGS. 1 and 2, in which:

FIGS. 1a-1f show the method steps for covering a NiTi stent with a NiTi layer, using micro-electroplating and sputter deposition;

FIG. 2 shows a scanning electron micrograph (internal view) of a NiTi stent, which is sputtered with a NiTi covering using the method described; and

FIGS. 3a-3e show the method steps for producing a NiTi stent with an inner layer and a NiTi coating layer using micro-electroplating and sputter deposition.

### DETAILED DESCRIPTION

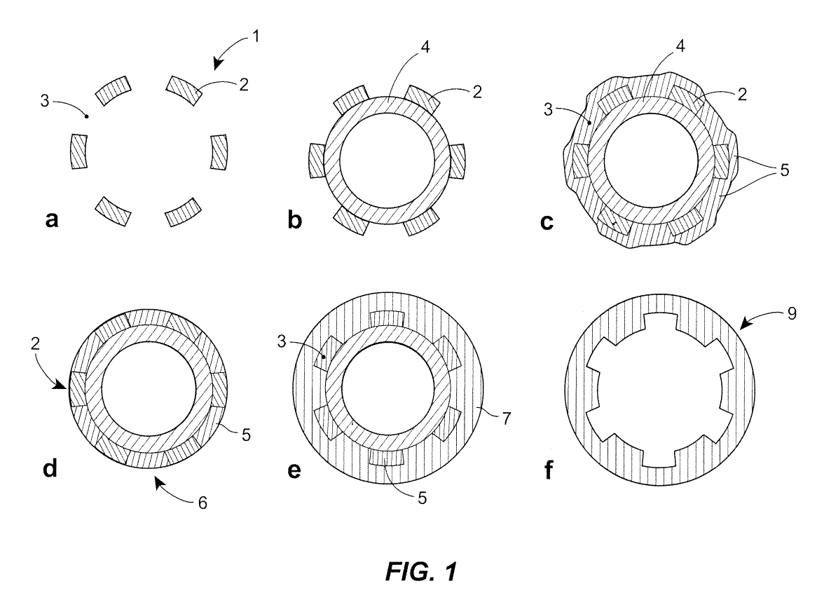

FIG. 1(a) shows a schematic section through a NiTi stent 1, whose wall 2 is interrupted by the apertures 3. This stent shows superelastic behavior at body temperature. By cooling the stent in the martensitic state, for example, by using a cooling spray or liquid nitrogen, the stent can be expanded and mounted on a cylindrical holder, represented here by a copper tube 4. In order to ensure a secure hold, the external diameter of the copper tube 4 is larger than the internal diameter of the NiTi stent. Subsequent warming of the stent to room temperature leads to form-fitting contact between the mounted stent and the substrate (FIG. 1b).

Subsequently, in a micro-electroplating process, a copper layer 5 is electrochemically deposited onto the copper substrate 4 as the sacrificial material. The deposition is continued until the whole stent is covered with copper and the sacrificial material fills the apertures 3 (FIG. 1c). The copper 5 covering the NiTi stent 1 is subsequently removed. This can be carried out manually with fine abrasive paper on a lathe. Copper is removed until the wall 2 of the NiTi stent in the applied Cu layer 5 is laid bare and a cylinder 6 of even wall thickness has been produced (FIG. 1d).

Now, in this case, a thin NiTi coating layer 7 is sputter deposited on the cylinder 6 thereby produced, said cylinder being formed by the stent 1 filled with Cu (FIG. 1c). In this case, the NiTi coating layer 7 bonds to the wall 2 of the stent. The total diameter increases by between 10 and 100 μm, corresponding to a wall thickness of the NiTi in the range of 5 μm to 50 μm. Finally, the cylindrical holder 4 and the sacrificial material 5 situated in the apertures 3 is removed by means of a selective etching medium, for example 40% HNO3, so that only the coated stent 9 remains (FIG. 1f). Selective etching is advantageously carried out using an acid pump which pumps the etching medium through the copper cylinder 4 and dissolves the core. Typical etching times are in the region of 10-30 minutes, and typical wall thicknesses of the copper tube are 0.5 mm, given an outer wall thickness of 5 mm.

In this way, in particular NiTi stents with a diameter of 4.5 mm and a wall thickness of 0.2 mm can be coated with a NiTi coating of 15 μm. It is possible with conventional photolithographic and wet chemical etching processes to structure the NiTi layer, for example using photoresist and a selective etching medium. In order to remove the sacrificial core, HNO3, Fe3Cl or ammonium peroxosulphate solution can be used as the etching medium.

The method can be implemented particularly advantageously with stents which have a diameter in the range of 100 μm to 100 mm, and particularly 1 mm to 36 mm, and a wall thickness in the range of 50 μm to 5 mm, and in particular 50 μm to 600 μm. The thickness of the NiTi coating is in the range of 1 μm to 100 μm, wherein the range of 5 μm to 50 μm is preferable.

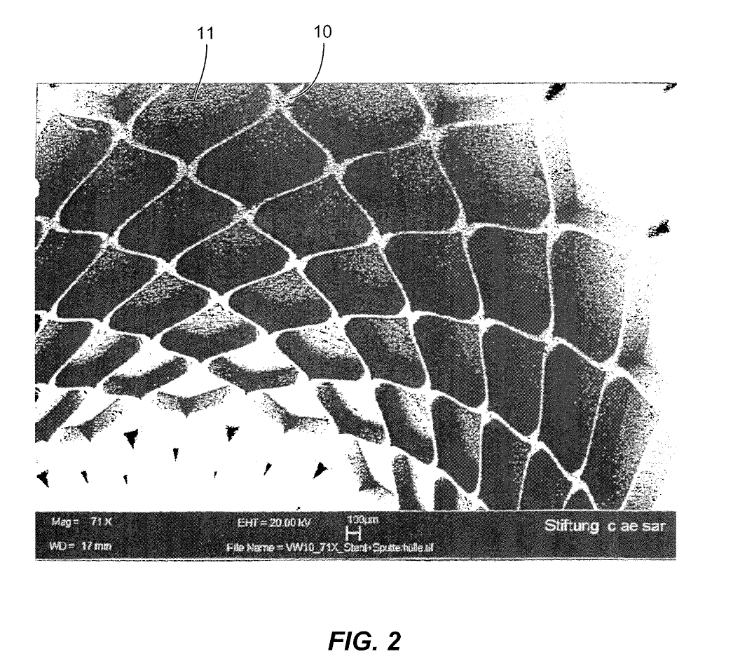

FIG. 2 shows an electron micrograph of the wall of a stent coated according to the disclosure. Clearly visible are the webs 10 of the net-like structure, whose meshes are covered on the outside with the NiTi coating 11. Shown below is a scale, indicating that the beams have a length of 100 μm. The wall thickness, that is, the width of the webs 10 is approximately in this region. Also visible is that the NiTi coating 11 covering the surface formed by the webs 10 is almost closed.

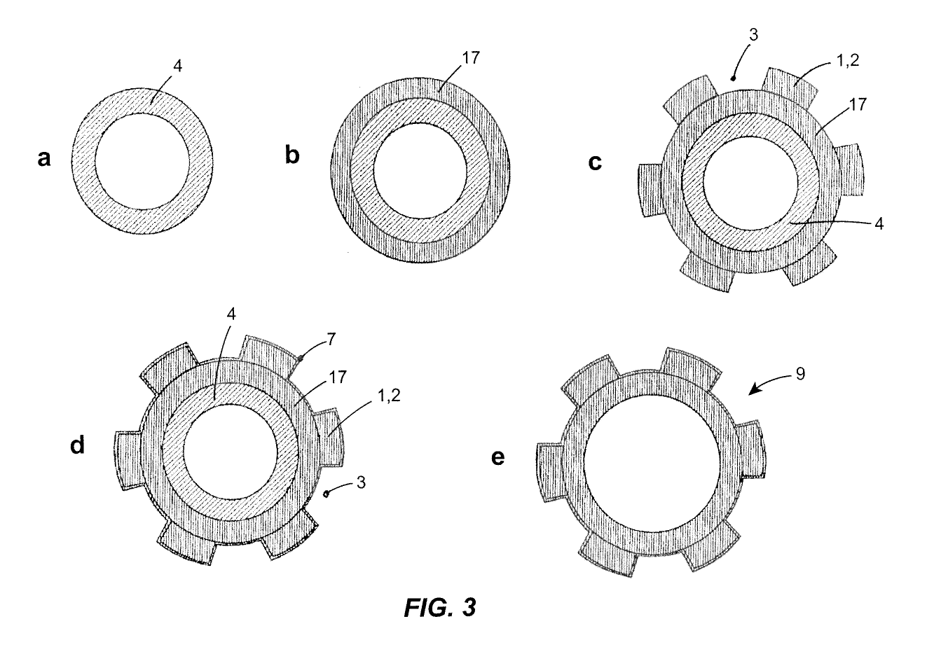

FIG. 3 shows the method steps for production of a NiTi stent 9 which, in the finished condition, additionally to the NiTi covering layer or coating layer 7 described above by reference to FIG. 1, hereinafter called the ‘first metal layer 7’, has a further layer 17. This further layer 17 is hereinafter called the ‘second metal layer 17’ and is preferably made from a metal, in particular a nickel-titanium alloy. In the method shown in FIG. 3, like that shown in FIG. 1, micro-electroplating and sputtering deposition can be used.

FIG. 3a shows schematically and in detail a section through a substrate 4 which, in this exemplary case, is cylindrical and serves as a holder. In a preferred embodiment of the method shown in FIG. 3, this holder can be made from a copper tube 4 wherein the copper material serves as sacrificial material for the later micro-electroplating. It is clear that the disclosure is not restricted to cylindrical substrates 4; rather, the shape of the substrate should be adapted to the shape of the stent 9 that is to be made.

In the next method step, a metal layer 17 is formed on the outside of the cylindrical substrate 4 shown in FIG. 3(a), preferably a NiTi layer, as shown in FIG. 3(b). Suitable methods can be used for this, such as a vapor deposition method or sputter deposition. Naturally, it is also conceivable that the second metal layer 17 is drawn over the cylindrical substrate 4 as a sleeve. Preferably, the method used should be designed to form the second metal layer 17 on the outside of the substrate 4 such that the layer thickness of the second metal layer 17 can be predetermined. Depending on the planned use of the finished stent 9, the second metal layer 17 has a thickness in the range of 1 μm to 10 mm, and preferably 5 μm to 50 μm.

Subsequently, a commercially available stent 1 is drawn over the second metal layer 17 mounted on the cylindrical holder 4. FIG. 3(c) shows schematically a section through a NiTi stent 1, whose wall 2 is interrupted by the apertures 3. This stent 1 shows superelastic behavior at body temperature. By cooling the stent in the martensitic condition, for example, by use of a cooling spray or in liquid nitrogen, the stent 1 can be expanded and mounted on the second metal layer 17 formed on the cylindrical holder 4. In order to ensure a secure hold, the outer diameter of the second metal layer 17 is preferably greater than the inner diameter of the NiTi stent 1.

Subsequent warning of the stent 1 to room temperature leads to a form-fitting contact between the mounted stent 1 and the second metal layer 17 (FIG. 3c).

Thereafter, in this case, a thin NiTi coating layer 7 is sputter deposited on the outside of the mounted stent 1 (FIG. 3d). In this case, the NiTi coating layer 7 bonds, on the one hand, to the wall 2 of the stent and, on the other hand, to the sections of the second metal layer 17 that are laid bare in the apertures 3. In the process, the overall diameter increases by an amount in the range of 10 μm to 100 μm, which corresponds to a wall thickness of the first metal layer 7 in the range of 5 μm to 50 μm.

Finally, the cylindrical holder 4 is removed by means of a selective etching medium, for example, 40% HNO3, such that only the stent 9 shown in FIG. 3(e) remains, said stent having the NiTi covering layer or coating layer 7 and the inner NiTi layer 17. The selective etching is advantageously carried out anew with the use of an acid pump which pumps the etching medium through the copper cylinder 4 and dissolves the core. Typical etching times are in the range of 10 mins to 30 mins and typical wall thicknesses of the copper tube are 0.5 mm, with an outer diameter of 5 mm.

In this way, in particular, NiTi stents with a diameter of 4.5 mm and a wall thickness of 0.2 mm can be coated with a NiTi outer covering of 15 μm and a NiTi inner covering in the range of 1 μm to 10 mm.

With current photolithographic and wet chemical etching methods it is possible to structure the NiTi outer layer 7 that has grown on or possibly also the NiTi inner layer 17, for example, using a photoresist and a selective etching agent. In order to remove the sacrificial core, HNO3, Fe3Cl or ammonium peroxosulphate solution can be used as the selective etching agent.

A stent 9 which has an inner metal film 17 as shown in FIG. 3(e) is characterised in that, it has, inter alia, an extremely smooth inner surface so that the body fluid can flow through the stent in its implanted state with particularly low resistance and, in particular, without turbulence. This also further reduces the risk of embolism. In addition, a stent with an inner metal film has advantages with regard to secure positioning of the implant in the body of the patient, since the problem of tensions, etc. arising in the implant can be reduced. It is also conceivable that drugs, etc. could be incorporated in the inner film, which is preferably a metal film, but can also be made from a plastics or a polymer composition, and that in the implanted condition of the stent, these could be continuously released over a long period.

It is a fundamental principle that—due to the thin film technology used for producing the layers—both the covering metal film and the inner metal film are very pure and therefore are particularly biocompatible.

The disclosure is not limited to the special embodiments illustrated in the figures, and in particular the tube-shaped implants shown. Rather, the method according to the disclosure can also be applied accordingly to flat tissue structures. Such pieces of tissue can be brought into the final form of the implant after their treatment, for example, by means of suitable shaping.

The disclosure is also not restricted to the production of stents. The method according to the disclosure can also be used for vascular implants such as filters, stent grafts or occlusion instruments. Furthermore, in place of a NiTi inner layer, a layer made from plastics or a polymer composition can be used.

1. Method for applying a coating layer to a tubular intraluminal implant, in particular to a vascular support, wherein the surface of the implant is perforated by a plurality of apertures, and wherein the coating layer is produced by deposition of material onto the surface of the implant, comprising
in a first step, pushing the implant onto a cylindrical holder,
in a second step, depositing a sacrificial material on the surface of the implant until the deposited sacrificial material at least almost fills the apertures,
in a third step, depositing the coating layer onto the surface of the implant provided with sacrificial material, and
in a fourth step, removing the cylindrical holder and the sacrificial material situated in the apertures.

2. Method according to claim 1, and, in an intermediate step between the second step and the third step, treating the surface of the implant provided with sacrificial material, wherein the sacrificial material is at least partially removed from outside.

3. Method according to claim 2, and removing the sacrificial material to such an extent that the surface of the implant, is freed from the sacrificial material.

4. Method according to claim 1, and applying the coating layer as an uninterrupted structure, wherein the dimension of the apertures is smaller by an order of magnitude than the apertures situated in the surface of the implant.

5. Method according to claim 1, and wherein a film of metal, is applied as the coating layer.

6. Method according to claim 5, wherein a nickel-titanium alloy is sputter deposited.

7. Method according to claim 1, wherein the sacrificial material is deposited in a galvanic method.

8. Method according to claim 1, wherein the cylindrical holder and the sacrificial material are removed by a selective acid treatment.

9. Method according to claim 1, wherein the cylindrical holder is made of metal.

10. Method according to claim 1, wherein the implant is pushed onto the holder while being stretched and is then relaxed.

11. Endoluminal implant, comprising a mesh structure and a first metal layer, the mesh structure being bonded throughout, with material contact, to the first metal layer.

12. Implant according to claim 11, wherein the first metal layer is configured as a coating layer which surrounds an outside of the mesh structure the implant.

13. Implant according to claim 12, wherein the coating layer comprises a nickel-titanium alloy.

14. Implant according to claim 11, wherein the mesh structure of the implant is a conventional stent with a diameter in the range of 100 μm to 100 mm.

15. Implant according to claim 14, wherein the stent has a wall thickness in the range of 50 μm to 5 mm.

16. Implant according to claim 11, wherein the first metal layer has a thickness in the range of 1 μm to 100 μm.

17. Implant according to claim 11, wherein the implant also comprises a second metal layer which is configured as a layer surrounding the inside of the mesh structure of the implant.

18. Implant according to claim 17, wherein the second metal layer lies against the inside of the mesh structure throughout.

19. Implant according to claim 17, wherein the second metal layer is bonded at least in sections to the first metal layer with material contact.

20. Implant according to claim 17, wherein the second metal layer comprises a nickel-titanium alloy.

21. Implant according to claim 17, wherein the second metal layer has a thickness in the range of 1 μm to 10 mm.

22. Method for producing an endoluminal implant, comprising bonding at least one layer to a mesh structure by means of one of a PVD process or a CVD process.

23. Method according to claim 22, and depositing a first layer of the at least one layer on an outside of the mesh structure and bonded thereto with material contact.

24. Method according to claim 23, and arranging a second layer of the at least one layer on an inside of the mesh structure, and depositing the first layer onto the outside of the mesh structure such that at least in sections, the second metal layer is bonded to the first metal layer with material contact.

25. Method according to claim 1, wherein the sacrificial material is copper.

26. Method according to claim 3, wherein the surface of the implant that is freed from sacrificial material comprises a mesh structure.

27. Method according to claim 5, wherein the film of metal is a shape memory alloy.

28. Method according to claim 5, wherein the film of metal is applied by vapor deposition.

29. Method according to claim 6, wherein the nickel-titanium alloy is Nitinol.

30. Method according to claim 9, wherein the metal is copper.

31. Implant according to claim 14, wherein the diameter is in the range of 1 mm to 36 mm.

32. Implant according to claim 15, wherein the wall thickness is in the range of 50 μm to 600 μm.

33. Implant of claim 16, wherein the thickness is in the range of 5 μm to 50 μm.

34. Implant according to claim 21, wherein the thickness is in the range of 5 μm to 50 μm.

35. Method according to claim 22, wherein the at least one layer is a metal layer.
